(19) United States
(12) Patent Application Publication
(10) Pub. No.: US 2011/0112382 A1
(43) Pub. Date: May 12, 2011

(54) SYSTEMS AND METHODS FOR COMBINED PHYSIOLOGICAL SENSORS

(75) Inventors: Youzhi Li, Longmont, CO (US); Bo Chen, Louisville, CO (US); Edward M. McKenna, Boulder, CO (US); Paul Stanley Addison, Edinburgh (GB)

(73) Assignee: Nellcor Puritan Bennett LLC, Boulder, CO (US)

(21) Appl. No.: 12/944,950

(22) Filed: Nov. 12, 2010

Related U.S. Application Data
(60) Provisional application No. 61/260,734, filed on Nov. 12, 2009.

(51) Int. Cl. A61B 5/00

(52) U.S. Cl. 600/301

(57) ABSTRACT

Systems and methods are provided for monitoring the physiological state of a subject. One or more physiological parameters of a subject may be determined from a photoplethysmograph (PPG) signal or signals obtained using at least one PPG sensor. In some embodiments, an electrical physiological signal (EPS) sensor may be located in or near a PPG sensor. A sensor configuration including both PPG sensors and EPS sensors may be advantageously used to detect a PPG signal or signals in combination with one or more EPS signal or signals. To reduce potential interference between an EPS sensor and a PPG sensor, fiber-optic input and output lines may be used to transmit optical signals from light generating circuitry and light detecting circuitry. In some embodiments, the generating and detecting circuitry may be located remotely from one another and may further be located remotely from the EPS sensor, PPG sensor, or both.

### CROSS-REFERENCE TO RELATED APPLICATION

This application claims priority to U.S. Provisional Patent Application No. 61/260,734, entitled “SYSTEMS AND METHODS FOR COMBINED PHYSIOLOGICAL SENSORS,” filed Nov. 12, 2009, which is hereby incorporated by reference herein in its entirety.

### SUMMARY

The present disclosure is related to signal processing systems and methods, and more particularly, to systems and methods for detecting one or more physiological characteristics of a subject using one or more sensors with combined photoplethysmographic (PPG) and electrical physiological parameter measurement capabilities.

A physiological sensor device may include a first sensor configured to receive an electrical signal of a subject and a second sensor configured to transmit to and receive from the subject an optical signal. For example, the first sensor may be an electrical physiological signal (EPS) sensor such as an electroencephalograph (EEG) sensor configured to receive an EEG signal, although it will be understood that any suitable EPS sensor may be used. The second sensor may be, for example, a PPG sensor such as an optical sensor (e.g., an oximetry sensor). In an embodiment, the second sensor may include one or more optical apertures configured to transmit and receive optical signals. The generated optical signal may include light of a single wavelength or multiple wavelengths. The second sensor may be coupled with circuitry for generating optical signals and converting received optical signals into electrical signals. The circuitry may be disposed remotely from the first sensor to reduce electrical interference between the circuitry and the first sensor. In some embodiments, components of the generating circuitry and detecting circuitry may be located remotely from one another and the generating circuitry and detecting circuitry may further be located remotely from the first sensor, the second sensor, or both. One or more fiber-optic lines may be used to transmit optical signals between the sensor and the generating circuitry and detecting circuitry.

The methods and systems of the present disclosure will be illustrated with reference to the monitoring of a physiological signal (e.g., a PPG signal or EPS signal); however, it will be understood that the disclosure is not limited to monitoring physiological signals and is usefully applied within a number of signal monitoring settings. Those skilled in the art will recognize that the present disclosure has wide applicability to other signals including, but not limited to, other biosignals (e.g., electrogastrogram, heart rate signals, pathological sounds, ultrasound, or any other suitable biosignal), condition monitoring signals, fluid signals, electrical signals, sound and speech signals, chemical signals, any other suitable signal, or any combination thereof.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 depicts a block diagram of a subject monitoring system sensor structure according to an illustrative arrangement;

FIG. 2 depicts a block diagram of a subject monitoring system according to an illustrative arrangement; and

FIG. 3 is a flowchart depicting an illustrative process for monitoring at least two different physiological parameters of a subject.

### DETAILED DESCRIPTION

Monitoring the physiological state of a subject, for example, by determining, estimating, and/or tracking one or more physiological parameters of the subject, may be of interest in a wide variety of medical and non-medical applications. Knowledge of a subject's physiological characteristics (e.g., through a determination of one or more physiological parameters such as blood pressure, oxygen saturation, and presence of specific heart conditions) can provide short- and long-term benefits to the subject, such as early detection and/or warning of potentially harmful conditions, diagnosis and treatment of illnesses, and/or guidance for preventative medicine.

Physiological parameters of a subject can be determined from a plethysmograph signal or a photoplethysmograph (PPG) signal, and such a signal can be obtained from a subject using a sensor. For example, a plethysmograph signal can be obtained from a subject using a sensor in the form of a pressure transducer that may be fastened to the subject's wrist area. Alternatively, or additionally, a PPG signal can be obtained using a PPG sensor in the form of an optical sensor that is clipped or fastened to a digit, appendage (e.g., an ear), or other part of the subject (the term “digit” refers herein to a toe or finger of a subject), such as the forehead. Such a PPG sensor may be used to emit and detect light that is used to determine the blood oxygen saturation of a subject.

Further, in an embodiment, a second PPG sensor may be affixed to a subject, and the combination of these two PPG sensors may allow for the determination of the subject's blood pressure, for example, using continuous non-invasive blood pressure (CNIBP) techniques. For example, in an arrangement, two PPG-based optical sensors can be used. One of these sensors may be used to determine the blood oxygen saturation of the subject, and the other sensor may be used alone or in combination to determine an estimate of the blood pressure of the subject via non-invasive techniques.

The use of PPG sensors, for example, for the measurement of oxygen saturation, blood pressure, and/or other physiological parameters may be complimented by the measurement of one or more other electrical physiological signal or signals. Electrical physiological signals (abbreviated EPS hereon) may include electroencephalographic (EEG) signals, electrocardiography (ECG or EKG) signals, electromyography (EMG) signals, or any other electrical physiological signal. For example, in an arrangement, an EPS sensor (e.g., an electrode) may be placed in or near each PPG sensor. For example, in an arrangement, each PPG sensor may be an optical sensor (e.g., a pulse oximetry sensor), and an EPS sensor may be placed within the housing of each of these PPG sensors. In general, a sensor configuration including both PPG sensors and EPS sensors may be advantageously used to detect a PPG signal or signals in combination with one or more EPS signal or signals, and may provide a range of useful information regarding a subject. For example, in an arrangement, one or more physiological parameters of a subject may be determined using PPG sensors (such as pulse oximetry sensors, CNIBP sensors) combined with EPS sensors to produce weighted biosignal information. In an arrangement, measurements made by each of these PPG sensors may be combined with measurements made by EPS sensors (e.g., EPS electrodes) to, for example, be used as a gating signal for determining a subject oxygen saturation level. In an arrangement, a filtering process may be used to, for example, trigger an ensemble averaging of at least two of the measured PPG signals, which may improve the derivation of physiological and/or biosignal parameters.

In an arrangement, a PPG sensor may be affixed to a subject. As described above, this PPG sensor may correspond to a pulse oximetry sensor (and may be used as a single sensor to determine a blood oxygen saturation level, and/or as one of two sensors in tandem to determine a subject blood pressure). The PPG sensor may emit light that is passed through or reflected by the tissue of a subject and detected by a detector. The light passed through or reflected by the tissue may be selected to be of one or more wavelengths that are absorbed by the subject's blood in an amount representative of the amount of the blood constituent present in the blood. The amount of light passed through or reflected by the tissue varies in accordance with the changing amount of blood constituent in the tissue and the related light absorption. Red and infrared wavelengths may be used because it has been observed that highly oxygenated blood will absorb relatively less red light and more infrared light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based on Lambert-Beer's law. The following notation will be used herein:

I(λ,t)=I₀(λ)exp(−(sβ₀(λ))+(1−s)βᵣ(λ))l(t)) (1)

where:
λ=wavelength;
t=time;
I=intensity of light detected;
I₀=intensity of light transmitted;
s=oxygen saturation;
β₀, βᵣ=empirically derived absorption coefficients; and
l(t)=a combination of concentration and path length from emitter to detector as a function of time.

Light absorption may be measured at two wavelengths (e.g., red and infrared (IR)), and then saturation may be calculated by solving for the “ratio of ratios” as follows.

1. First, the natural logarithm of (1) is taken (“log” will be used to represent the natural logarithm) for IR and Red

log I=log I₀−(sβ₀+(1−s)βᵣ)l (2)

2. (2) is then differentiated with respect to time

$$\frac{d log I}{d t} = - ( s {β}_{o} + ( 1 - s ) {β}_{r} ) \frac{d l}{d t} \qquad ( 3 )$$

3. Red (3) is divided by IR (3)

$$\frac{d log I ( {λ}_{R} ) / d t}{d log I ( {λ}_{IR} ) / d t} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} \qquad ( 4 )$$

### 4. Solving for s

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\frac{d log I ( {λ}_{R} )}{d t} ( {β}_{o} ( {λ}_{IR} ) - {β}_{r} ( {λ}_{IR} ) ) - \frac{d log I ( {λ}_{IR} )}{d t} ( {β}_{o} ( {λ}_{R} ) - {β}_{r} ( {λ}_{R} ) )}$$

Note in discrete time

$$\frac{d log I ( λ , t )}{d t} ≃ log I ( λ , {t}_{2} ) - log I ( λ , {t}_{1} )$$

### Using log A−log B=log A/B,

$$\frac{d log I ( λ , t )}{d t} ≃ log ( \frac{I ( {t}_{2} , λ )}{I ( {t}_{1} , λ )} )$$

So, (4) can be rewritten as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R \qquad ( 5 )$$

where R represents the “ratio of ratios.” Solving (4) for s using (5) gives

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) + {β}_{r} ( {λ}_{R} )} .$$

From (5), R can be calculated using two points (e.g., PPG maximum and minimum), or a family of points. One method using a family of points uses a modified version of (5). Using the relationship

$$\frac{d log I}{d t} = \frac{d I / d t}{I} \qquad ( 6 )$$

now (5) becomes

$$\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} = R \qquad ( 7 )$$

which defines a cluster of points whose slope of y versus x will give R where

x(t)=[I(t₂,λ_{IR})−I(t₁,λ_{IR})]I(t₁,λ_{R})

y(t)=[I(t₂,λ_{R})−I(t₁,λ_{R})]I(t₁,λ_{IR})

y(t)=Rx(t)

Once R is determined or estimated, for example, using the techniques described above, the blood oxygen saturation can be determined or estimated using any suitable technique for relating a blood oxygen saturation value to R. For example, blood oxygen saturation can be determined from empirical data that may be indexed by values of R, and/or it may be determined from curve fitting and/or other interpolative techniques.

In an arrangement, at least two PPG sensors may be affixed to a subject. As described above, these PPG sensors may correspond to pulse oximetry sensors, and may be used to determine a CNIBP of a subject. Each sensor may be positioned at a different respective location on a subject's body to estimate the blood pressure and/or other related biosignal parameters of the subject from a measured signal or signals. In an arrangement, a reference point of a measured signal may be identified (and this reference point may correspond to a reference “feature,” such as a leading or trailing edge of the signal, or the location of a signal peak or valley), and the elapsed time, denoted T, between the arrival times of this reference point at the two sensors (e.g., pulse oximetry sensors) may be determined. An estimate of the subject's blood pressure, p, may then be determined from any suitable relationship between the blood pressure and T. For example, in an arrangement, the following mathematical relation may be used to determine an estimate of subject blood pressure from the elapsed time

p=a+b·ln(T),

where a and b are constants that may be determined from a calibration process and may be dependent on the nature of the subject and signal detector that are, for example, affixed to the subject. Once calibration has been completed, for example, using a non-invasive blood pressure device, an equation similar or identical to the one above can be used to determine a subject blood pressure. The equation above is meant to be illustrative, and any other suitable equation (or equations) may also be used to derive an estimated subject blood pressure. Further, blood pressure estimates may be computed on a continuous or periodic basis. Alternatively or additionally, in an embodiment, T may be taken as the difference in time between a reference point on an ECG signal and a reference point on a PPG signal. The pulse transit time may be used instead of the above difference in arrival times of two PPG signals, for example, to determine a blood pressure measurement value.

EPS measurements may be very sensitive to other forms of electrical interference, such as electrical drive or data signals from an adjacent or nearby electrical device. For example, an EEG electrode on a subject may be susceptible to interference from the electrical circuitry of a nearby PPG sensor. In order to reduce potential interference between an EPS sensor and a nearby PPG sensor, fiber-optic input and output lines may be used to transmit the light signals needed for the PPG measurement from the generating and detecting circuitry, which may be located away from the actual PPG sensor and the nearby EPS sensor. In some embodiments, the generating and detecting circuitry may be located remotely from one another to reduce electrical interference. For example, the light detecting circuitry may be located proximate the PPG sensor or may be substantially embedded in the PPG sensor, and the light generating circuitry may be located remotely from the PPG sensor and the EPS sensor. As another example, the light generating circuitry may be located proximate the PPG sensor or may be substantially embedded in the PPG sensor, and the light detecting circuitry may be located remotely from the PPG sensor and the EPS sensor. Either configuration may be preferable, for example, depending on whether the generating and detecting circuitry produce different levels of electrical interference. As discussed above, in some embodiments, both the generating and detecting circuitry may be located remotely from the PPG sensor and the EPS sensor. In such an embodiment, the generating and detecting circuitry may also be located remotely from one another. Fiber-optic input and output lines may be used to transmit the light signals needed for the PPG measurement from the generating and detecting circuitry, whether the circuitry is positioned locally or remotely.

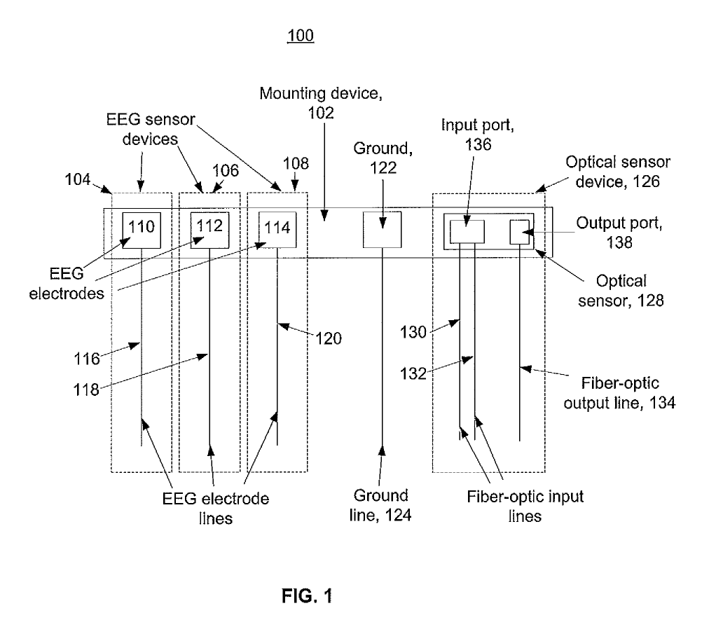

FIG. 1 depicts a block diagram of a subject monitoring system sensor structure 100 according to an illustrative arrangement. Sensor structure 100 may include a plurality of sensor devices disposed on a mounting device 102. Mounting device 102 may be configured to be mounted on a subject's head, and may, for example, be a headband or included as part of a headband. In other arrangements, the sensor structure 100 may be configured to be mounted elsewhere on the subject. Sensor structure 100 may include a plurality of EPS sensor devices. In the depicted arrangement, sensor structure 100 includes three EEG sensor devices 104, 106, and 108, but in other arrangements, fewer or more sensor devices may be included, and sensor devices may be included to measure other electrical physiological signals of the subject, such as ECG or EMG signals. In some arrangements, sensor structure 100 may be able to measure a plurality of electrical physiological signals of the subject. For example, sensor structure 100 may include sensors for sensing EEG and EMG signals. Each EEG sensor device may include an electrode (110, 112, and 114) mounted on the mounting device 102, and may include an electrode line (116, 118, and 120) electrically connecting the electrode to one or more sensor input ports (not shown). The electrodes 104-108 may be disposed to contact the subject in order to better sense the relevant EPS. In some arrangements, the electrodes 104-108 may be disposed at various locations on the subject's head. For example, one electrode may be disposed in the center of the subject's forehead, one electrode may be disposed above one eyebrow, and one electrode may be disposed at the temple closest to the one eyebrow. In some arrangements, an EEG sensor device will function as a passive sensor. Optionally, one or more EEG sensor devices may be used to measure a physiological signal that requires actuation of the sensor device. For example, if an impedance of the subject is to be measured, at least one of the electrode lines 116-120 may be driven with an input current or voltage, and the output currents and/or voltages may be measured at the other electrodes. In some arrangements, sensor structure 100 may include a ground 122 mounted on mounting device 102. Ground 122 may provide a ground for the EEG sensor devices 104-108, and may be electrically connected to one or more ground input ports (not shown) via ground line 124. In some arrangements, ground 122 may be separate from the mounting device 102, and may be disposed on the subject's head, such as on the bridge of the subject's nose. In other embodiments, the ground 122 may be disposed elsewhere on the subject. For example, ground 122 may be disposed at a digit, appendage (e.g., an ear), any other suitable part of the subject, or any combination thereof that may provide a suitable electrical ground.

Sensor structure 100 may include at least one optical sensor device 126 (e.g., a PPG or oximeter sensor device) mounted on mounting device 102. Optical sensor device 126 may include an optical sensor 128 with one or more fiber-optic input lines 130-132 and a fiber-optic output line 134. Optical sensor 128 may be disposed on the subject in order to perform oximetry measurements and/or blood pressure measurements. As discussed above, sensitive measurements such as EEG measurements may be subject to interference from nearby electrical activity. Hence, placing the electrical-optical conversion circuitry of the optical sensor system away from the sensor structure 100 and using fiber-optic lines to transport the optical signals may reduce the amount of interference or noise EEG electrodes 110-114 detect. Fiber-optic input lines 130-132 may transport optical signals from one or more emitters (not shown) to the site of interest. The transported optical signals may be coherent light, such as light from lasers, or may be noncoherent light. In some arrangements, the fiber-optic input lines 130 and 132 may each transport a different wavelength of light. For example, fiber-optic line 130 may transport red light, and fiber-optic line 132 may transport IR light. In other arrangements, one or more fiber-optic lines 130-132 may transport multiple wavelengths of light. For example, red and IR light may be mixed together and transported via a single fiber-optic line. In these arrangements, only one fiber-optic input line may be included.

The fiber-optic input lines 130-132 may transport light to one or more input ports 136 located in optical sensor 128. Input port 136 may include one or more exit apertures (not shown) for enabling light to exit the optical sensor 128. Each fiber-optic input line may have its own exit aperture, or multiple fiber-optic input lines may share one or more exit apertures.

Light that exits input port 136 may be transmitted into the subject, and reflected from one or more internal surfaces or structures. In some arrangements, the reflected light signals may contain information about one or more physiological signals. Optical sensor 128 may include one or more output ports 138 for receiving reflected light signals from the subject. Output port 138 may include one or more entrance apertures (not shown) for receiving the reflected light signals. In some arrangements, each entrance aperture may be configured to receive a particular light wavelength. For example, an entrance aperture may include a filter for filtering particular light wavelengths. In other arrangements, an entrance aperture may be configured to receive light of multiple wavelengths. The entrance apertures in output port 138 may be coupled to one or more fiber-optic output lines 134, which may transport the received reflected light signals to one or more receivers (not shown).

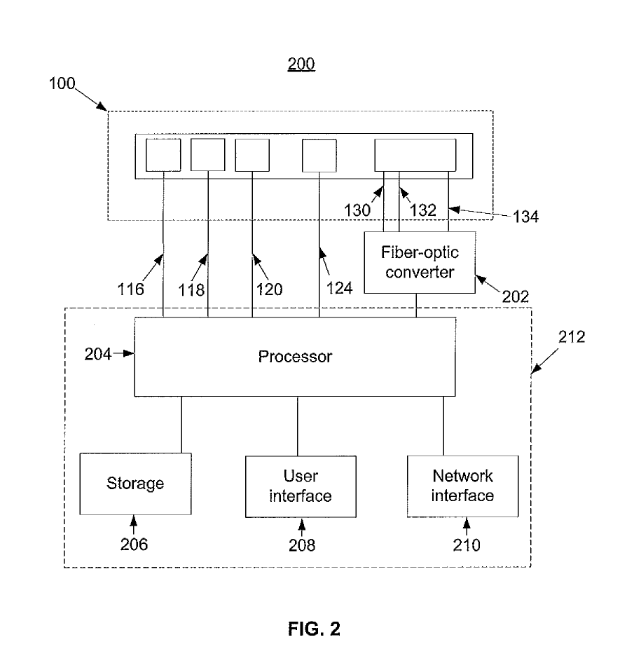

FIG. 2 depicts a block diagram of a subject monitoring system 200 according to an illustrative arrangement. Monitoring system 200 includes sensor structure 100, described above in relation to FIG. 1. Monitoring system 200 may also include a fiber-optic converter 202, a processor 204, storage 206, user interface 208, and network interface 210. Electrode lines 116-120 and ground line 124 may electrically connect electrodes 110-114 and ground 122 to processor 204. Fiber-optic input lines 130-132 and fiber-optic output line 134 may transport light to and from fiber-optic converter 202. In some embodiments, one or more of processor 204, storage 206, user interface 208, and network interface 210 may be disposed within a monitor 212. As depicted, the fiber-optic converter 202 is not included as part of sensor structure 100 or monitor 212. For example, the fiber-optic converter 202 may be incorporated into cabling or an interconnect located between sensor structure 100 and monitor 212. In other arrangements, a portion or all of the fiber-optic converter 202 may be included in monitor 212 or in sensor structure 100 (e.g., as part of optical sensor 128).

Fiber-optic converter 202 may include one or more light emitters and one or more light detectors (not shown). The light emitters in fiber-optic converter 202 may be coupled to the fiber-optic input lines 130-132. For example, input line 130 may be coupled to one light emitter, and input line 132 may be coupled to another light emitter. In certain arrangements, a particular input line may be coupled to more than one light emitter. For example, two or more light emitters may emit light of different wavelengths, which may be mixed and coupled to the input line. In other arrangements, one light emitter may be coupled to more than one input line.

The one or more light detectors in fiber-optic converter 202 may also be coupled to the fiber-optic output line 134. For example, output line 134 may be coupled to one light detector in fiber-optic converter 202. In other arrangements, output line 134 may be coupled to more than one light detector in converter 202.

The light emitters and detectors in fiber-optic converter 202 may be configured to convert electrical signals into light signals, and vice-versa. For example, the light emitters in converter 202 may convert an electrical signal into light of a particular wavelength, and the light detectors in converter 202 may convert light of particular wavelengths into electrical signals with particular frequencies or amplitudes. In some arrangements, each light detector in converter 202 may be configured to be responsive only to light of a certain wavelength. In other arrangements a particular light detector may be sensitive to a number of light wavelengths.

In an embodiment, light emitters and detectors, such as the light emitters and detectors in fiber-optic converter 202, may be located remotely from one another. For example, in some embodiments a fiber-optic converter may include either an emitter or a detector. At least two fiber-optic converters may be provided (not shown) in which one fiber-optic converter includes a light emitter and another fiber-optic converter includes a light detector. The fiber-optic converters may then be positioned remotely from one another. Alternatively, or additionally, at least one of the light emitters or detectors may be located separately from a fiber-optic converter.

Fiber-optic converter 202 may be communicatively coupled with processor 204. For example, processor 204 may provide the electrical drive signals for light emitters in the converter 202 to convert into light, and may receive converted electrical signals from light detectors in the converter 202. In other arrangements, fiber-optic converter 202 may be independently capable of generating drive signals for its light emitters, and the processor 204 may only supply instructions to the converter 202 while receiving converted electrical signals from the converter light detectors.

Processor 204 may be any suitable software, firmware, and/or hardware, and/or combinations thereof for processing signals or for performing processing tasks related to various PPG and EPS measurements. For example, processor 204 may be configured to process received electrical signals to determine relevant physiological parameters. Processor 204 may include one or more hardware processors (e.g., integrated circuits), one or more software modules, computer-readable media such as memory, firmware, or any combination thereof. Processor 204 may, for example, be a computer or may be one or more chips (i.e., integrated circuits). Processor 204 may perform any suitable signal processing, such as any suitable band-pass filtering, adaptive filtering, closed-loop filtering, and/or any other suitable filtering, and/or any combination thereof.

Processor 204 may also be linked to storage 206 or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 204 to, for example, store data corresponding to physiological parameters. Storage 206 may include one or more storage devices, and may include one or more databases containing relevant physiological data. Storage 206 may also store operating instructions and software for processor 204.

Processor 204 may also be linked to user interface 208 and/or network interface 210. User interface 208 may include any suitable output device such as, for example, one or more medical devices (e.g., a medical monitor that displays various physiological parameters, a medical alarm, or any other suitable medical device that either displays physiological parameters or uses the output of processor 204 as an input), one or more display devices (e.g., monitor, PDA, mobile phone, any other suitable display device, or any combination thereof), one or more audio devices, one or more printing devices, any other suitable output device, or any combination thereof. User interface 208 may also include one or more user input devices, such as a keyboard or mouse, with which a user may input information or instructions for processor 204. Network interface 210 may link processor 204 with one or more networks.

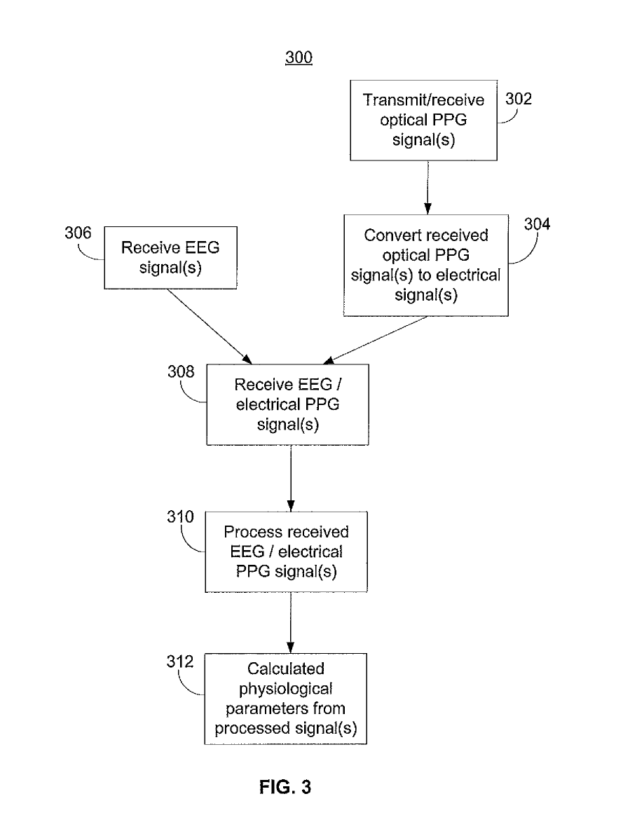

FIG. 3 is a flowchart depicting an illustrative process 300 for monitoring at least two different physiological parameters of a subject. At step 302, one or more optical signals may be transmitted and/or received by, for example, fiber-optic converter 202 (FIG. 2), which may then convert the received optical signals (e.g., PPG or oximetry signals) to one or more electrical signals at step 304. In an embodiment, the transmitter and receiver may be located remotely from one another. For example, a first fiber-optic converter having a transmitter and a second fiber-optic converter having a receiver may be provided, or at least one of the transmitter or receiver may be located remotely from a fiber-optic converter. At step 306, one or more EEG electrical signals or other electrical physiological signals may be received by, for example, EEG sensor devices 104-108 (FIG. 1). At step 308, the EEG electrical signals and electrical PPG signals may be received by, for example, processor 204. At step 310, the received signals may be processed by, for example, processor 204. The processing performed by processor 204 may include noise removal, analog-to-digital conversion, or any other analog or digital signal processing. At step 312, one or more physiological parameters may be calculated or determined from the processed signals by, for example, processor 204. Examples of calculated physiological parameters may include consciousness indices, pulse rate, blood oxygen saturation, blood pressure, respiratory rate, respiratory effort, vasomotion, vascular compliance, cardiac output, or any other suitable physiological parameter.

The foregoing is merely illustrative of the principles of this disclosure and various modifications can be made by those skilled in the art without departing from the scope and spirit of the disclosure. The above described embodiments are presented for purposes of illustration and not of limitation. The present disclosure also can take many forms other than those explicitly described herein. Accordingly, it is emphasized that the disclosure is not limited to the explicitly disclosed methods, systems and apparatuses, but is intended to include variations to and modifications thereof which are within the spirit of the following claims.

What is claimed is:

1. A physiological sensor device comprising:
a first sensor configured to receive a first electrical signal of a subject; and
a second sensor configured to transmit to or receive from the subject a first optical signal, the second sensor comprising:
an optical aperture configured to transmit or receive the first optical signal, wherein the second sensor is capable of being coupled with circuitry for generating the first optical signal or converting the first optical signal into a second electrical signal and wherein the circuitry is disposed remote from the first sensor to reduce electrical interference between the circuitry and the first sensor.

2. The device of claim 1, wherein the optical aperture is disposed near the first sensor.

3. The device of claim 1, wherein the first sensor is an EEG sensor, and the first electrical signal is an EEG signal.

4. The device of claim 1, wherein the second sensor is a PPG sensor.

5. The device of claim 1, wherein the optical aperture and the circuitry are coupled by at least one fiber optic line.

6. The device of claim 1, wherein the first optical signal is transmitted by the second sensor into the subject, and wherein the second sensor is configured to receive a second optical signal from the subject.

7. The device of claim 6, wherein the first optical signal is generated by the circuitry, and wherein the second optical signal is converted into the second electrical signal by the circuitry.

8. The device of claim 6, wherein the first optical signal includes light of at least one wavelength.

9. The device of claim 6, wherein the first optical signal includes light of a plurality of wavelengths.

10. The device of claim 1, wherein the circuitry is included as part of the second sensor.

11. The device of claim 1, wherein the circuitry is included in a monitor remote from the second sensor.

12. A method for receiving physiological signals, comprising:
receiving a first electrical signal of a subject with a first sensor; and
transmitting a first optical signal into the subject with a second sensor, the second sensor comprising:
an optical aperture configured to transmit the first optical signal, wherein the second sensor is capable of being coupled with circuitry for generating the first optical signal and wherein the circuitry is disposed remote from the first sensor to reduce electrical interference between the circuitry and the first sensor.

13. The method of claim 12, wherein the optical aperture is disposed near the first sensor.

14. The method of claim 12, wherein the first sensor is an EEG sensor, and the first electrical signal is an EEG signal.

15. The method of claim 12, wherein the second sensor is a PPG sensor.

16. The method of claim 12, wherein the optical aperture and the circuitry are coupled by at least one fiber optic line.

17. The method of claim 12, wherein the first optical signal includes light of at least one wavelength.

18. The method of claim 12, wherein the first optical signal includes light of a plurality of wavelengths.

19. The method of claim 12, wherein the circuitry is included in a monitor remote from the second sensor.

20. A method for receiving physiological signals, comprising:
receiving a first electrical signal of a subject with a first sensor; and
receiving a first optical signal of the subject with a second sensor, the second sensor comprising:
an optical aperture configured to receiving the first optical signal, wherein the second sensor is capable of being coupled with circuitry for converting the first optical signal into a second electrical signal, wherein the circuitry is disposed remote from the first sensor to reduce electrical interference from the circuitry and the first sensor.
